(19) United States
(12) Patent Application Publication
(10) Pub. No.: US 2022/0249028 A1
(43) Pub. Date: Aug. 11, 2022

(54) BIOLOGICAL SIGNAL ESTIMATION DEVICE, BIOLOGICAL SIGNAL ESTIMATION METHOD, AND RECORDING MEDIUM STORING BIOLOGICAL SIGNAL ESTIMATION PROGRAM

(71) Applicant: NEC Corporation, Minato-ku, Tokyo (JP)

(72) Inventor: Kazuki IHARA, Tokyo (JP)

(73) Assignee: NEC Corporation, Minato-ku, Tokyo (JP)

(21) Appl. No.: 17/623,655

(22) PCT Filed: Jul. 16, 2019
(86) PCT No.: PCT/JP2019/027941

§ 371 Date: Dec. 29, 2021

(51) Int. Cl. A61B 5/00

(52) U.S. Cl. CPC A61B 5/725; A61B 2560/0475; A61B 5/7207; A61B 5/0004

(57) ABSTRACT

Accurate, low cost detection of biological signals can be achieved by a biological signal estimation device provided with: a first measurement unit for measuring a first signal generated in a living body; a second measurement unit for measuring a second signal generated in the living body; a comparison unit for comparing characteristics of the first signal and the second signal; and an estimation unit for estimating a biological signal in the living body by performing signal processing on the first signal and the second signal on the basis of the result of comparison of the characteristics by the comparison unit.

### TECHNICAL FIELD

The present invention relates to a technology for detecting a biological signal due to a pulse or the like.

### BACKGROUND ART

A method of detecting a biological signal due to a pulse or the like by using a sensor attached to a subject and analyzing an emotion, a health condition, or the like of the subject based on the detected biological signal is widely known. For example, it is possible to monitor a stress occurrence situation in the daily life of the subject by detecting the detected signal over a long period of time and analyzing the emotion of the subject. In order to accurately monitor the stress occurrence situation, it is necessary to accurately detect the biological signal over a long period of time.

Such detection of the biological signal due to a pulse or the like is performed by, for example, detecting a variation in the blood volume of the blood vessel due to pulsation in the subject as a photoplethysmography (PPG) signal representing a change in light intensity due to a change in absorbance.

In this case, a motion (body motion) of the body of the subject in daily life affects the blood flow, and as a result, noise (hereinafter, may be referred to as “motion artifact”) is added to the detected (measured) PPG signal. That is, since the detected PPG signal usually includes a motion artifact signal (noise signal), it is necessary to remove the motion artifact signal in order to improve accuracy in detection of the biological signal.

As a method for removing such a motion artifact signal, for example, a method using a three-axis acceleration sensor or the like has been known. However, the method using a three-axis acceleration sensor or the like causes an increase in costs. Therefore, expectations for a technology for implementing accurate, low-cost detection of a biological signal of a subject are increasing.

As a technology related to such a technology, Patent Literature 1 discloses a device including a first signal source, first and second signal detection devices, and a processor. In this device, the first signal source is arranged at a first position and emits a light beam onto the surface of the subject. The first signal detection device is arranged at a second position and detects a first signal associated with the light beam reflected by the subject. The second signal detection device is arranged at a third position and detects a second signal associated with the light beam reflected by the subject. Then, the processor determines the biological signal of the subject based on the first and second signals.

In addition, Patent Literature 2 discloses a pulse wave detection device including first and second piezoelectric sensors and pulse wave information acquisition means. In this device, the first piezoelectric sensor is arranged on the artery of a subject, and detects pulsation of the artery and pressure fluctuation of a body surface due to a motion. The second piezoelectric sensor is arranged in the vicinity away from the artery of the subject, and detects pressure fluctuation of the body surface due to the motion. Then, the pulse wave information acquisition means acquires information regarding a pulse wave from signals detected by the first and second piezoelectric sensors.

In addition, Patent Literature 3 discloses an electronic device including a pulse sensor that irradiates a subject with irradiation light and receives the irradiation light that is reflected from the subject to detect a pulse.

### CITATION LIST

### Patent Literature

[PTL 1] JP 2018-534031 A
[PTL 2] JP 2000-051164 A
[PTL 3] JP 2017-142867 A

### SUMMARY OF INVENTION

### Technical Problem

In the devices disclosed in Patent Literatures 1 and 2, the accuracy in detection of the biological signal of the subject is improved by removing the motion artifact signal based on two signals obtained in different measurement environments without using, for example, the three-axis acceleration sensor. However, in the devices disclosed in Patent Literatures 1 and 2, which of two obtained signals is regarded as a signal for motion artifact detection and which is regarded as a signal for biological signal detection (that is, a signal from which a signal regarded as the signal for motion artifact detection is to be removed) is determined in advance. Therefore, in a case where an installation state (for example, a positional relationship between the sensor and the blood vessel) such as a position where the signal detection device (sensor) is arranged is not appropriate, the accuracy in detection of the biological signal of the subject may decrease, and it cannot be said that it is sufficient to implement accurate, low-cost detection of the biological signal of the subject. In addition, Patent Literature 3 does not mention solving such a problem. A main object of the present invention is to provide a biological signal estimation device and the like that solve this problem.

### Solution to Problem

A biological signal estimation device according to one mode of the present invention includes: first measurement means configured to measure a first signal generated in a living body; second measurement means configured to measure a second signal generated in the living body; comparison means configured to compare characteristics of the first and second signals; and estimation means configured to estimate a biological signal in the living body by performing signal processing on the first and second signals based on a result of the comparison of the characteristics performed by the comparison means.

In another aspect for achieving the object, a biological signal estimation method according to one mode of the present invention includes: measuring, by first measurement means, a first signal generated in a living body; measuring, by second measurement means, a second signal generated in the living body; comparing, by an information processing device, characteristics of the first and second signals; and estimating, by the information processing device, a biological signal in the living body by performing signal processing on the first and second signals based on a result of the comparison of the characteristics.

In still another aspect for achieving the object, a biological signal estimation program according to one mode of the present invention causes a computer to implement: a comparison function of comparing characteristics of a first signal measured by first measurement means and a second signal measured by second measurement means, the first and second signals being generated in a living body; and an estimation function of estimating a biological signal in the living body by performing signal processing on the first and second signals based on a result of the comparison of the characteristics obtained by the comparison function.

The present invention can also be implemented by a non-volatile computer-readable recording medium storing the biological signal estimation program (computer program).

### Advantageous Effects of Invention

The present invention makes it possible to implement accurate, low-cost detection of a biological signal.

### BRIEF DESCRIPTION OF DRAWINGS

FIG. 1 is a block diagram illustrating a configuration of a biological signal estimation device 10 according to a first example embodiment of the present invention.

FIG. 2 is a diagram illustrating a waveform of a photoplethysmography (PPG) signal measured by a measurement unit 11 according to the first example embodiment of the present invention and a frequency spectrum of the PPG signal calculated by a comparison unit 12.

FIG. 3 is a diagram illustrating a waveform of a PPG signal measured by the measurement unit 11 according to the first example embodiment of the present invention and a waveform of a pulse signal estimated by an estimation unit 13.

FIG. 4 is a diagram illustrating a physical structure of a biological signal estimation device 10 according to the first example embodiment of the present invention and a mode in which the biological signal estimation device 10 is attached to a living body 20.

FIG. 5 is a flowchart illustrating an operation of the biological signal estimation device 10 according to the first example embodiment of the present invention.

FIG. 6 is a diagram illustrating a mode in which a large number of measurement units 11 according to the first example embodiment of the present invention are arranged in a lattice pattern.

FIG. 7 is a block diagram illustrating a configuration of a biological signal estimation device 40 according to a second example embodiment of the present invention.

FIG. 8 is a block diagram illustrating a configuration of an information processing device 900 capable of implementing the biological signal estimation device 10 according to the first example embodiment or the biological signal estimation device 40 according to the second example embodiment of the present invention.

### EXAMPLE EMBODIMENT

Hereinafter, example embodiments of the present invention will be described in detail with reference to the drawings.

### First Example Embodiment

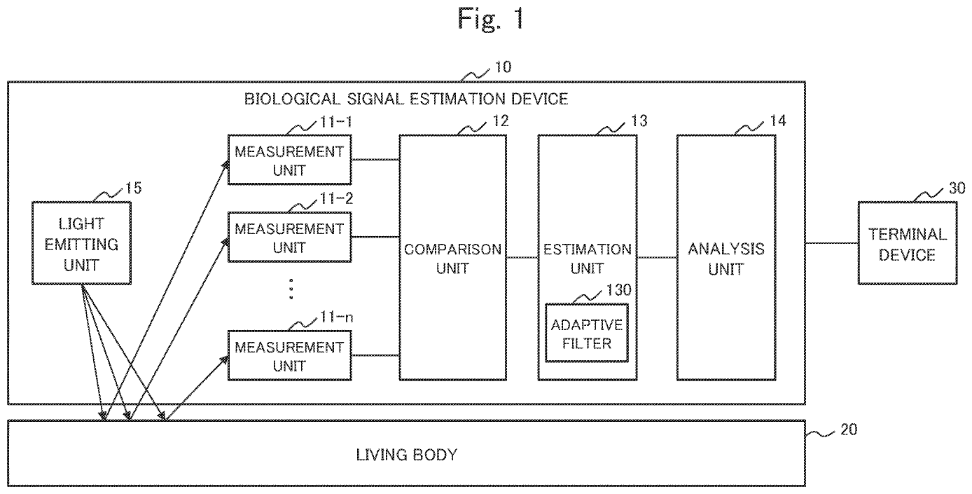

FIG. 1 is a block diagram illustrating a configuration of a biological signal estimation device 10 according to a first example embodiment of the present invention. The biological signal estimation device 10 is a device that detects (measures), for example, a biological signal representing a pulse of a living body 20 (subject) (hereinafter, may be referred to as a “pulse signal” in the present application), and analyzes an emotion of the living body 20 based on the detected pulse signal.

The biological signal estimation device 10 includes measurement units 11-1 to 11-n (n is an arbitrary integer equal to or more than 2 or more), a comparison unit 12, an estimation unit 13, an analysis unit 14, and a light emitting unit 15. In the present application, hereinafter, the measurement units 11-1 to 11-n may be collectively referred to as a measurement unit 11.

The measurement units 11-1 to 11-n are pulse sensors that detect pulse signals at different portions in the living body 20. The measurement unit 11 detects the pulse signal of the living body 20 by measuring an intensity signal indicated by light reflected by the living body 20 after the light emitting unit 15 that is, for example, a light emitting diode (LED), emits light toward the living body 20. More specifically, the measurement unit 11 detects a variation in a blood volume of a blood vessel due to pulsation in the living body 20 as a photoplethysmography (PPG) signal representing a change in light intensity due to a change in absorbance. Since a method by which the measurement unit 11 detects such a PPG signal is an existing technology used in a general photoplethysmography device, a detailed description of the method is omitted in the present application.

The PPG signal is a signal obtained as a sum of blood whose volume fluctuates due to pulsation in the living body 20, blood whose volume does not fluctuate due to pulsation, venous blood, and light absorption in a body tissue. Among them, light absorption in the blood whose volume fluctuates due to pulsation is expressed as an alternating current (AC) component in the PPG signal, and other light absorption is expressed as a direct current (DC) component in the PPG signal.

The PPG signal measured by the measurement unit 11 includes a motion artifact signal generated due to a motion in daily life of the living body 20 as noise.

The measurement unit 11 inputs the measured PPG signal to the comparison unit 12.

The comparison unit 12 compares characteristics of the PPG signals input from the measurement units 11-1 to 11-n. For example, the comparison unit 12 obtains frequency characteristics (frequency spectra) of the PPG signals input from the measurement unit 11 by using fast Fourier transform (FFT), and then compares the obtained frequency characteristics of the PPG signals.

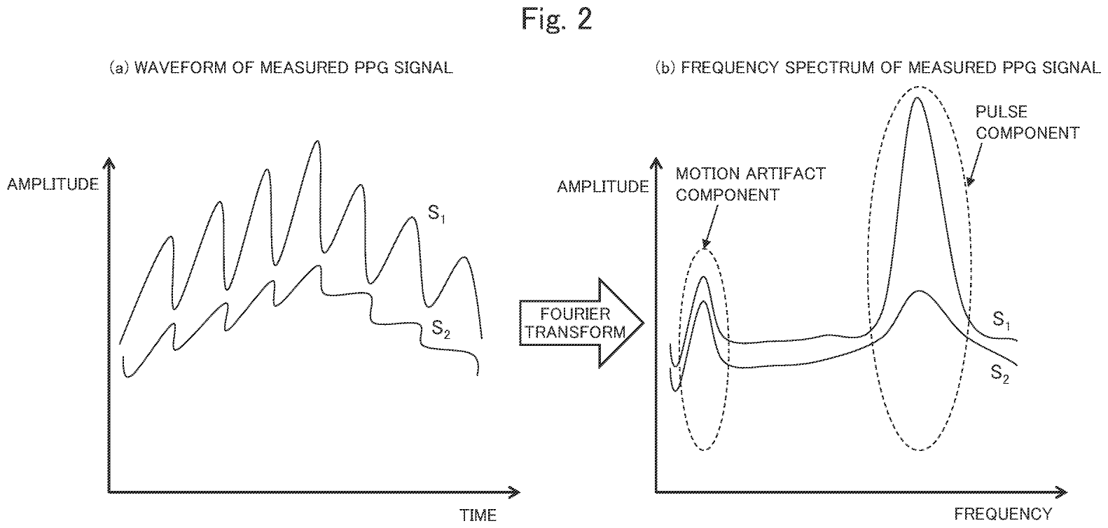

FIG. 2 is a diagram illustrating a waveform of the PPG signal measured by the measurement unit 11 according to the present example embodiment and the frequency spectrum of the PPG signal calculated by the comparison unit 12. In the example illustrated in FIG. 2, the biological signal estimation device 10 includes two measurement units 11-1 and 11-2. A PPG signal S₁ (first signal) illustrated in FIG. 2(a) is a signal measured by the measurement unit 11-1, and a PPG signal S₂ (second signal) is a signal measured by the measurement unit 11-2. In FIG. 2(a), a horizontal axis represents time, and a vertical axis represents an amplitude of the signal.

The comparison unit 12 calculates the frequency spectra of the PPG signals S₁ and S₂ as illustrated in FIG. 2(b) by performing Fourier transform on the PPG signals S₁ and S₂ illustrated in FIG. 2(a). In FIG. 2(b), a horizontal axis represents the frequency of the signal, and a vertical axis represents the amplitude (signal intensity) of the signal.

The comparison unit 12 performs comparison of a signal intensity of a frequency component (second frequency component) derived from a motion artifact with respect to that of a frequency component (first frequency component) derived from the pulse for the PPG signals S₁ and S₂ based on a result of calculating the frequency spectrum as illustrated in FIG. 2(b). It is known that a frequency derived from the pulse is generally about 40 to 200 hertz (Hz) and a frequency derived from the motion artifact is generally equal to or less than 10 Hz, and it is assumed that information regarding the frequency derived from the pulse and the frequency derived from the motion artifact is provided to the comparison unit 12.

In the example illustrated in FIG. 2(b), a ratio of the signal intensity of the frequency component derived from the motion artifact to that of the frequency component derived from the pulse is higher in the PPG signal S₂ than in the PPG signal S₁. That is, in this case, a proportion of the component of the motion artifact is higher in the PPG signal S₂ than in the PPG signal S₁, and this is because the measurement unit 11-1 is arranged at a position closer to the blood vessel in the living body 20 than the measurement unit 11-2 is.

The comparison unit 12 determines that the PPG signal S₁ is regarded as a signal for pulse signal detection (that is, a signal from which a signal regarded as a signal for motion artifact detection to be removed) and the PPG signal S₂ is regarded as the signal for motion artifact detection based on a result of the comparison described above.

In a case where the number of measurement units 11 included in the biological signal estimation device 10 is two (measurement units 11-1 and 11-2), the comparison unit 12 is operated as described above. However, in a case where the biological signal estimation device 10 includes three or more measurement units 11, the comparison unit 12 may be operated as follows. That is, the comparison unit 12 obtains the frequency spectra of the three or more PPG signals measured by the three or more measurement units 11 as described above, and compares the obtained frequency spectra of the PPG signals. The comparison unit 12 specifies at least one of the three or more PPG signals as the signal for pulse signal detection, and specifies at least one of the three or more PPG signals as the signal for motion artifact detection, based on the comparison result.

For example, in a case where the biological signal estimation device 10 includes four measurement units 11 (measurement units 11-1 to 11-4), the comparison unit 12 performs comparison of the signal intensity of the frequency component derived from the motion artifact with respect to that of the frequency component derived from the pulse for each of the four PPG signals obtained by the measurement units 11-1 to 11-4.

In this case, for example, the comparison unit 12 may specify a PPG signal having the lowest signal intensity ratio indicated by the comparison result among the four PPG signals as the signal for pulse signal detection, specify a PPG signal having the highest signal intensity ratio as the signal for motion artifact detection, and specify the other two PPG signals as signals that are not used in pulse signal estimation processing performed by the estimation unit 13. Alternatively, for example, the comparison unit 12 may specify a PPG signal having the lowest signal intensity ratio and a PPG signal having the second lowest signal intensity ratio indicated by the comparison result among the four PPG signals as the signals for pulse signal detection, and may specify a PPG signal having the highest signal intensity ratio and a PPG signal having the second highest signal intensity ratio as the signals for motion artifact detection.

In the example illustrated in FIG. 2, the comparison unit 12 inputs information indicating that the PPG signal S₁ is the signal for pulse signal detection and the PPG signal S₂ is the signal for motion artifact detection to the estimation unit 13 together with the PPG signals S₁ and Sz.

The estimation unit 13 performs signal processing of removing the PPG signal S₂ from the PPG signal S₁ based on the information input from the comparison unit 12.

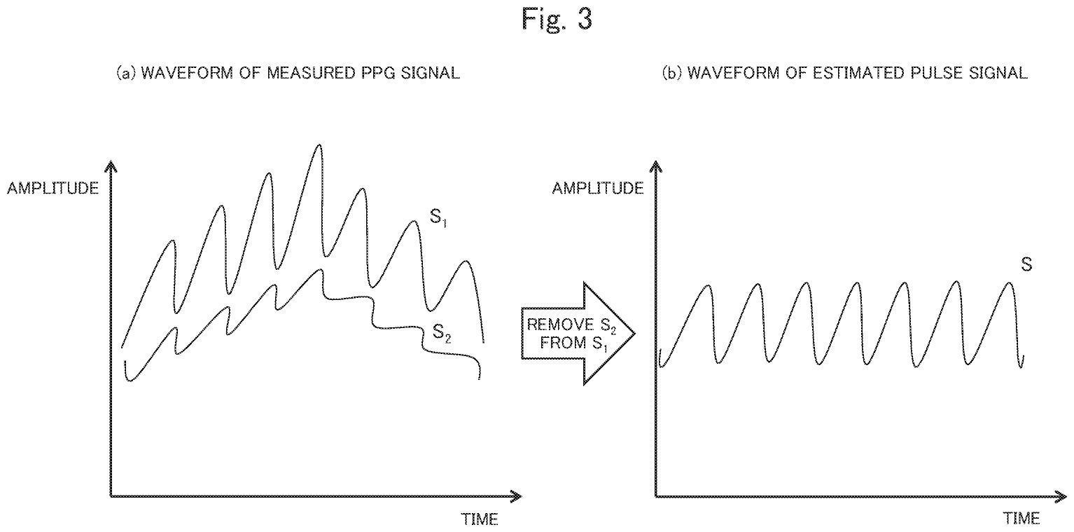

FIG. 3 is a diagram illustrating a waveform of the PPG signal measured by the measurement unit 11 according to the example embodiment and a waveform of the pulse signal estimated (generated) by the estimation unit 13. As illustrated in FIG. 3, the estimation unit 13 performs signal processing of removing, from the PPG signal S₁, a signal component corresponding to the PPG signal S₂ included in the PPG signal S₁, thereby estimating a pulse signal S representing the original pulse generated in the living body 20.

The estimation unit 13 performs signal processing of removing the PPG signal S₂ from the PPG signal S₁ by using, for example, an adaptive filter 130 illustrated in FIG. 1. Since the adaptive filter 130 is an existing technology using an optimization algorithm that uses an objective function that is a criterion for determination of optimum filter performance (for example, performance of minimizing a noise component of an input) to determine how to correct a filter coefficient in the next iteration (feedback), a detailed description thereof is omitted in the present application.

Alternatively, the estimation unit 13 may estimate the pulse signal S by, for example, simply performing signal processing of subtracting the PPG signal S₂ from the PPG signal S₁ without including the adaptive filter 130. In this case, the estimation unit 13 may perform weighting on the PPG signal S₂ by using a value indicating a ratio of the PPG signal S₁ to the PPG signal S₂ regarding a magnitude of a motion artifact component included in the PPG signal S₁ and S₂ illustrated in FIG. 2(b).

Furthermore, in a case where the biological signal estimation device 10 includes three or more measurement units 11, when the comparison unit 12 specifies a plurality of PPG signals as the signal for pulse signal detection and the signal for motion artifact detection, the estimation unit 13 may perform statistical computation such as obtaining an average on the plurality of PPG signals. For example, in a case where the comparison unit 12 specifies two PPG signals as the signals for pulse signal detection, the estimation unit 13 may use an average value of the two PPG signals when estimating the pulse signal. In addition, in a case where the comparison unit 12 specifies the two PPG signals as the signals for motion artifact detection, the estimation unit 13 may use an average value of the two PPG signals when estimating the pulse signal.

The estimation unit 13 inputs the pulse signal S estimated by the signal processing described above to the analysis unit 14.

The analysis unit 14 analyzes an emotion of the living body 20 based on the waveform of the pulse signal S or the like input from the estimation unit 13. Since the analysis unit 14 can use an existing technology of analyzing the emotion of the living body 20 based on the waveform of the pulse signal S or the like, a detailed description thereof is omitted in the present application.

The analysis unit 14 transmits a result of analyzing the emotion of the living body 20 to a terminal device illustrated in FIG. 1. However, a terminal device 30 is an information processing device such as a personal computer used when a user refers to information output from the biological signal estimation device 10 or when the user inputs information to the biological signal estimation device 10.

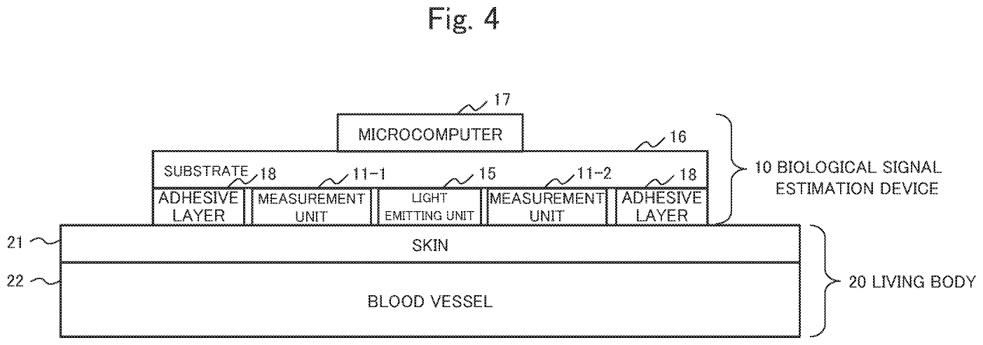

FIG. 4 is a diagram illustrating a physical structure of the biological signal estimation device 10 according to the present example embodiment and a mode in which the biological signal estimation device 10 is attached to the living body 20. However, in the example illustrated in FIG. 4, the biological signal estimation device 10 includes two measurement units 11-1 and 11-2.

The biological signal estimation device 10 is adhered to a surface of a skin 21 of the living body 20 by an adhesive layer 18. The measurement units 11-1 to 11-2 measure the PPG signals indicated by reflected light, which is emitted from the light emitting unit 15 toward the living body 20, from the skin 21 and a blood vessel 22 of the living body 20.

A microcomputer 17 is a logic circuit such as a large scale integration (LSI), and receives the PPG signals measured by the measurement units 11-1 and 11-2 via a substrate 16. The substrate 16 may be formed of, for example, a stretchable material for flexible attachment of the biological signal estimation device 10 to the living body 20. The microcomputer 17 includes at least some of the comparison unit 12, the estimation unit 13, the analysis unit 14, and a communication function (not illustrated) for communicating with an external device such as the terminal device 30. At least some of the comparison unit 12, the estimation unit 13, and the analysis unit 14 may be provided in a server device or the like capable of communicating with the biological signal estimation device 10. That is, for example, in a case where the microcomputer 17 includes the comparison unit 12 and the estimation unit 13, the biological signal estimation device 10 transmits the pulse signal S estimated by the estimation unit 13 to the server device including the analysis unit 14. Then, the server device may perform processing of analyzing the emotion of the living body 20 based on the received pulse signal S.

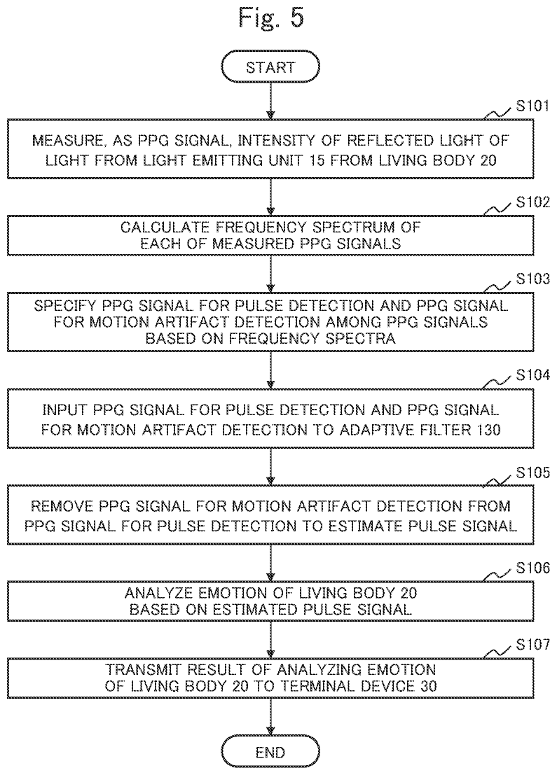

Next, an operation (processing) of the biological signal estimation device 10 according to the present example embodiment will be described in detail with reference to the flowchart of FIG. 5.

The measurement units 11 measure, as the PPG signals, the intensities of the reflected light of the light from the light emitting unit 15 from the living body 20 (step S101). The comparison unit 12 calculates the frequency spectrum of each of the PPG signals measured by the measurement units 11 (step S102). The comparison unit 12 specifies the PPG signal for pulse detection and the PPG signal for motion artifact detection among the PPG signals based on the frequency spectra of the PPG signals (step S103).

The estimation unit 13 inputs the PPG signal for pulse detection and the PPG signal for motion artifact detection specified by the comparison unit 12 to the adaptive filter 130 (step S104). The estimation unit 13 removes the PPG signal for motion artifact detection from the PPG signal for pulse detection by using the adaptive filter 130 to estimate the pulse signal representing the original pulse generated in the living body 20 (step S105).

The analysis unit 14 analyzes the emotion of the living body 20 based on the pulse signal estimated by the estimation unit 13 (step S106). The analysis unit 14 transmits a result of analyzing the emotion of the living body 20 to the terminal device 30 (step S107), and the entire processing ends.

The biological signal estimation device 10 according to the present example embodiment can implement accurate, low-cost detection of the biological signal. This is because the biological signal estimation device 10 measures the first and second signals generated in the living body 20, compares the characteristics of the measured first and second signals, and estimates the biological signal in the living body 20 by performing signal processing on the first and second signals based on the comparison result.

Hereinafter, effects achieved by the biological signal estimation device 10 according to the present example embodiment will be described in detail.

There is a system that detects a biological signal due to a pulse or the like by using a sensor attached to a subject and analyzes an emotion, a health condition, or the like of the subject based on the detected biological signal. In such a system, since the detected biological signal usually includes a noise signal such as a motion artifact signal, it is necessary to remove the motion artifact signal or the like in order to improve accuracy in detection of the biological signal. A method of removing the motion artifact signal or the like includes, for example, a method using a three-axis acceleration sensor, but this method causes an increase in costs. In addition, a method of removing the motion artifact signal based on two signals obtained in different measurement environments as described in Patent Literatures 1 and 2 described above may also be used. However, in a case where an installation state of the sensor is not appropriate, the accuracy in detection of the biological signal may decrease. Therefore, there is a problem in implementing accurate, low-cost detection of the biological signal of the subject.

In order to solve such a problem, the biological signal estimation device 10 according to the present example embodiment includes the measurement units 11-1 to 11-n, the comparison unit 12, and the estimation unit 13, and is operated as described above with reference to FIGS. 1 to 5, for example. That is, the two or more measurement units 11 measure the first and second signals generated in the living body 20. The comparison unit 12 compares the characteristics of the first and second signals. Then, the estimation unit 13 estimates the biological signal in the living body 20 by performing signal processing on the first and second signals based on a result of the comparison of the characteristics performed by the comparison unit 12.

That is, the biological signal estimation device 10 according to the present example embodiment specifies the PPG signal for pulse detection and the PPG signal for motion artifact detection based on a result of comparing characteristics of first and second PPG signals measured by the measurement units 11, and then removes the PPG signal for motion artifact detection from the PPG signal for pulse detection to estimate the pulse signal generated in the living body 20. As a result, the biological signal estimation device 10 can implement accurate, low-cost detection of the pulse signal of the living body 20 regardless of an installation state of the biological signal estimation device 10.

The biological signal estimation device 10 according to the present example embodiment measures three or more PPG signals, and specifies at least one PPG signal for pulse detection and at least one PPG signal for motion artifact detection based on a result of comparing characteristics of the PPG signals. Then, in a case where a plurality of PPG signals for pulse detection or a plurality of PPG signals for motion artifact detection are specified, the biological signal estimation device 10 performs statistical computation such as average calculation on the plurality of PPG signals for pulse detection or the plurality of PPG signals for motion artifact detection. As described above, the biological signal estimation device 10 according to the present example embodiment can further improve accuracy in detection of the pulse signal of the living body 20 by using a number of PPG signals.

Furthermore, the biological signal estimation device 10 according to the present example embodiment may estimate a biological signal other than the pulse signal in the living body 20. For example, the fact that light absorption characteristics of blood vary depending on a state of the blood (concentrations of various blood components such as blood oxygen saturation) has been known. By using the fact, the biological signal estimation device 10 may estimate a biological signal indicating a state of the blood by measuring, as the PPG signal, reflected light obtained by irradiating the living body 20 with beams of light having different wavelengths from a plurality of light emitting units 15, for example. Also in this case, since the measured PPG signal includes noise such as the motion artifact, the biological signal estimation device 10 can accurately estimate the biological signal indicating the state of the blood by removing the noise based on the characteristics indicated by the PPG signals.

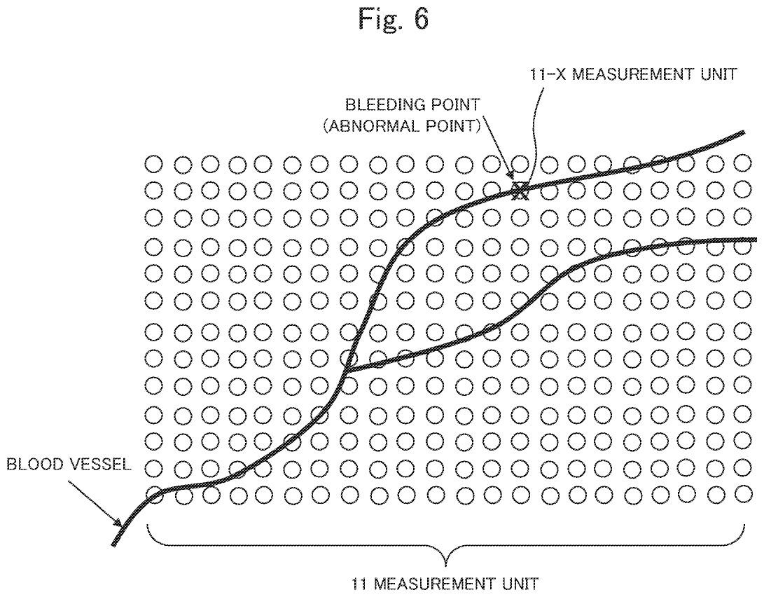

An analysis target of the analysis unit 14 according to the present example embodiment is not limited to the emotion of the living body 20, and various states of the living body 20 that is a human or an animal other than a human may be analyzed. For example, as illustrated in FIG. 6, the biological signal estimation device 10 may include a large number of measurement units 11 arranged in a lattice pattern to analyze an abnormal point (mutated point) such as a bleeding point in the blood vessel of the living body 20.

In the example of FIG. 6, there is an abnormal point in the blood vessel in the vicinity of a measurement unit 11-X. A signal mode is different between a portion of the blood vessel that is positioned downstream of the abnormal point and a non-abnormal portion of the blood vessel other than the portion positioned downstream of the abnormal point due to a biological reaction occurring at the abnormal point. Examples of the different signal modes include a change in volume change amount due to bleeding, an increase in concentration of an inflammation-derived substance, and an increase in concentration of a marker molecule due to malignant neoplasm. For example, in a case where bleeding occurs in the vicinity of the measurement unit 11-X, a mode in which the blood volume in the blood vessel due to pulsation is changed is different between a portion of the blood vessel positioned downstream of a bleeding point and a non-bleeding portion of the blood vessel other than the portion positioned downstream of the bleeding point. Therefore, in this case, a characteristic (for example, a frequency characteristic) of a biological signal estimated based on PPG signals measured by the measurement unit 11-X and a measurement unit 11 positioned downstream of the measurement unit 11-X is different from a characteristic of a biological signal estimated based on PPG signals measured by measurement units 11 other than the measurement unit 11-X and the measurement unit 11 positioned downstream of the measurement unit 11-X, which are arranged near the blood vessel. Therefore, the biological signal estimation device 10 can specify the bleeding point in the blood vessel of the living body 20 by specifying the measurement unit 11-X that the characteristic of the estimated biological signal is changed among the measurement units 11 arranged in the vicinity of the blood vessel. In the example illustrated in FIG. 6, the biological signal estimation device 10 can use a PPG signal measured by a measurement unit 11 arranged at a place away from the blood vessel as the PPG signal for the motion artifact detection, thereby increasing accuracy in specifying the bleeding point.

### Second Example Embodiment

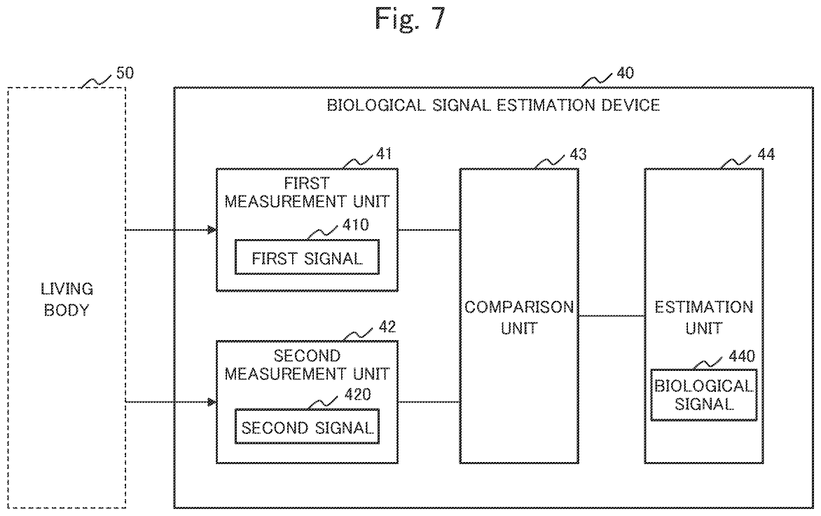

FIG. 7 is a block diagram illustrating a configuration of a biological signal estimation device 40 according to a second example embodiment of the present invention. The biological signal estimation device 40 includes a first measurement unit 41, a second measurement unit 42, a comparison unit 43, and an estimation unit 44.

The first measurement unit 41 measures a first signal 410 generated in a living body 50.

The second measurement unit 42 measures a second signal 420 generated in the living body 50.

The first measurement unit 41 and the second measurement unit 42 may measure PPG signals as the first signal 410 and the second signal 420, for example, similarly to the measurement unit 11 according to the first example embodiment described above.

The comparison unit 43 compares characteristics of the first signal 410 and the second signal 420. The comparison unit 43 may obtain frequency characteristics of the first signal 410 and the second signal 420, for example, similarly to the comparison unit 12 according to the first example embodiment described above. Similarly to the comparison unit 12, the comparison unit 43 may specify the first signal 410 as a signal for pulse signal detection and may specify the second signal 420 as a signal for motion artifact detection.

The estimation unit 44 estimates a biological signal 440 in the living body 50 by performing signal processing on the first signal 410 and the second signal 420 based on a result of the comparison of the characteristics performed by the comparison unit 43. For example, similarly to the estimation unit 13 according to the first example embodiment described above, the estimation unit 44 may perform signal processing of removing the second signal 420 specified as the signal for motion artifact detection from the first signal 410 specified as the signal for pulse signal detection.

The biological signal estimation device 40 according to the present example embodiment can implement accurate, low-cost detection of the biological signal. This is because the biological signal estimation device 40 measures the first and second signals generated in the living body 50, compares the characteristics of the measured first and second signals, and estimates the biological signal in the living body 50 by performing signal processing on the first and second signals based on the comparison result.

### Hardware Configuration Example

Each unit in the biological signal estimation device 10 illustrated in FIG. 1 or the biological signal estimation device 40 illustrated in FIG. 7 in each example embodiment described above can be implemented by dedicated hardware (HW) (electronic circuit). In FIGS. 1 and 7, at least the following components can be regarded as a functional (processing) unit (software module) of a software program.
Comparison units 12 and 43
Estimation units 13 and 44
Analysis unit 14

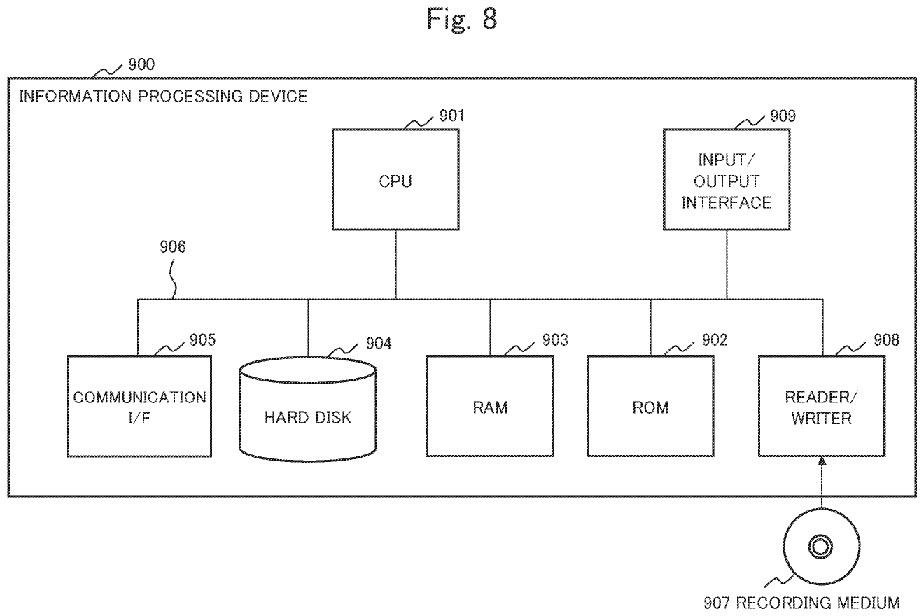

However, the division of the units illustrated in these drawings is a configuration for convenience of description, and various configurations can be assumed at the time of actual implementation. An example of a hardware environment in this case will be described with reference to FIG. 8.

FIG. 8 is a diagram illustrating an exemplary configuration of an information processing device 900 (computer) capable of implementing the biological signal estimation device 10 according to the first example embodiment or the biological signal estimation device 40 according to the second example embodiment of the present invention. That is, FIG. 8 illustrates a configuration of a computer (information processing device) capable of implementing the biological signal estimation devices 10 and 40 illustrated in FIGS. 1 and 7, and illustrates a hardware environment capable of implementing each function in the above-described example embodiments.

The information processing device 900 illustrated in FIG. 8 includes the following components, but does not include some of the following components in some cases.
Central processing unit (CPU) 901
Read only memory (ROM) 902
Random access memory (RAM) 903
Hard disk (storage device) 904
Communication interface 905 for communication with external device
Bus 906 (communication line)
Reader/writer 908 capable of reading and writing data stored in recording medium 907 such as compact disc read only memory (CD-ROM);
Input/output interface 909 such as monitor, speaker, or keyboard.

That is, the information processing device 900 including the above-described components is a general computer to which these components are connected via the bus 906. The information processing device 900 may include a plurality of CPUs 901 or may include a CPU 901 implemented by multiple cores.

The present invention described using the above-described example embodiments as examples supplies a computer program capable of implementing the following functions to the information processing device 900 illustrated in FIG. 8. The functions refer to the above-described configurations in the block configuration diagrams (FIGS. 1 and 7) referred to in the description of the example embodiments or the functions in the flowchart (FIG. 5). Further, the present invention is achieved by loading the computer program into the CPU 901 of the hardware and interpreting and executing the computer program. The computer program supplied into the device may be stored in a readable/writable volatile memory (RAM 903) or a nonvolatile storage device such as the ROM 902 or the hard disk 904.

Furthermore, in the above case, a general procedure can be adopted at present as a method of supplying the computer program into the hardware. Examples of the procedure include a method of installing the program in the device via various recording media 907 such as a CD-ROM, a method of downloading the program from the outside via a communication line such as the Internet, and the like. In such a case, the present invention can be regarded as being configured by a code constituting the computer program or the recording medium 907 storing the code.

The present invention has been described above using the above-described example embodiments as exemplary examples. However, the present invention is not limited to the above-described example embodiments. That is, the present invention can apply various modes that can be understood by those skilled in the art within the scope of the present invention.

Some or all of the above-described example embodiments can also be described as the following Supplementary Notes. However, the present invention illustratively described by the above-described example embodiments is not limited to the following.

(Supplementary Note 1)

A biological signal estimation device including:

first measurement means configured to measure a first signal generated in a living body;

second measurement means configured to measure a second signal generated in the living body;

comparison means configured to compare characteristics of the first and second signals; and

estimation means configured to estimate a biological signal in the living body by performing signal processing on the first and second signals based on a result of the comparison of the characteristics performed by the comparison means.

(Supplementary Note 2)

The biological signal estimation device according to supplementary note 1, in which the comparison means calculates frequency characteristics of the first and second signals, and then performs comparison of a signal intensity of a second frequency component with respect to that of a first frequency component for each of the first and second signals.

(Supplementary Note 3)

The biological signal estimation device according to supplementary note 1 or 2, in which the estimation means performs the signal processing of removing the second signal from the first signal.

(Supplementary Note 4)

The biological signal estimation device according to supplementary note 3, in which the estimation means performs the signal processing of subtracting the second signal from the first signal.

(Supplementary Note 5)

The biological signal estimation device according to supplementary note 3, in which the estimation means performs the signal processing using an adaptive filter on the first and second signals.

(Supplementary Note 6)

The biological signal estimation device according to any one of supplementary notes 1 to 4, in which the comparison means specifies at least one first signal and at least one second signal by comparing the characteristics of a plurality of signals measured by a plurality of measurement means including the first and second measurement means, the plurality of measurement means measuring signals generated in the living body.

(Supplementary Note 7)

The biological signal estimation device according to supplementary note 6, in which the estimation means performs statistical computation on a signal specified as the first signal and performs statistical computation on a signal specified as the second signal.

(Supplementary Note 8)

The biological signal estimation device according to any one of supplementary notes 1 to 7, in which the first and second measurement means measure the first and second signals including a pulse signal and a motion artifact signal generated in the living body, and

the estimation means estimates the pulse signal as the biological signal.

(Supplementary Note 9)

The biological signal estimation device according to any one of supplementary notes 1 to 8, further including analysis means configured to analyze a state of the living body based on the biological signal estimated by the estimation means.

(Supplementary Note 10)

The biological signal estimation device according to supplementary note 9, in which the analysis means analyzes an emotion of the living body.

(Supplementary Note 11)

The biological signal estimation device according to supplementary note 9, in which the analysis means analyzes an abnormal point in the living body.

(Supplementary Note 12)

The biological signal estimation device according to supplementary note 11, in which the analysis means specifies, as the abnormal point, a point where the first measurement means related to the biological signal whose characteristic is changed among a plurality of the biological signals is arranged, the plurality of the biological signals being estimated based on measurement results of a plurality of the first measurement means arranged in the vicinity of a blood vessel of the living body.

(Supplementary Note 13)

A biological signal estimation method including:

measuring, by first measurement means, a first signal generated in a living body;

measuring, by second measurement means, a second signal generated in the living body;

comparing, by an information processing device, characteristics of the first and second signals; and

estimating, by the information processing device, a biological signal in the living body by performing signal processing on the first and second signals based on a result of the comparison of the characteristics.

(Supplementary Note 14)

A recording medium storing a biological signal estimation program for causing a computer to implement:

a comparison function of comparing characteristics of a first signal measured by first measurement means and a second signal measured by second measurement means, the first and second signals being generated in a living body; and

an estimation function of estimating a biological signal in the living body by performing signal processing on the first and second signals based on a result of the comparison of the characteristics obtained by the comparison function.

### REFERENCE SIGNS LIST

10 biological signal estimation device
11-1 to 11-n measurement unit
12 comparison unit
13 estimation unit
130 adaptive filter
14 analysis unit
15 light emitting unit
16 substrate
17 microcomputer
18 adhesive layer
20 living body
21 skin
22 blood vessel
30 terminal device
40 biological signal estimation device
41 first measurement unit
410 first signal
42 second measurement unit
420 second signal
43 comparison unit
44 estimation unit
440 biological signal
50 living body
900 information processing device
901 CPU
902 ROM
903 RAM
904 hard disk (storage device)
905 communication interface
906 bus
907 recording medium
908 reader/writer
909 input/output interface

What is claimed is:

1. A biological signal estimation device comprising:
a first measurement device configured to measure a first signal generated in a living body;
a second measurement device configured to measure a second signal generated in the living body;
at least one memory storing a computer program; and
at least one processor configured to execute the computer program to
compare characteristics of the first and second signals; and
estimate a biological signal in the living body by performing signal processing on the first and second signals based on a result of the comparison of the characteristics performed.

2. The biological signal estimation device according to claim 1, wherein the processor is configured to execute the computer program to
calculate frequency characteristics of the first and second signals, and then perform comparison of a signal intensity of a second frequency component with respect to that of a first frequency component for each of the first and second signals.

3. The biological signal estimation device according to claim 1, wherein the processor is configured to execute the computer program to perform the signal processing of removing the second signal from the first signal.

4. The biological signal estimation device according to claim 3, wherein the processor is configured to execute the computer program to perform the signal processing of subtracting the second signal from the first signal.

5. The biological signal estimation device according to claim 3, wherein the processor is configured to execute the computer program to perform the signal processing using an adaptive filter on the first and second signals.

6. The biological signal estimation device according to claim 1, wherein the processor is configured to execute the computer program to specify at least one first signal and at least one second signal by comparing the characteristics of a plurality of signals measured by a plurality of measurement devices including the first and second measurement devices, the plurality of measurement devices measuring signals generated in the living body.

7. The biological signal estimation device according to claim 6, wherein the processor is configured to execute the computer program to perform statistical computation on a signal specified as the first signal and perform statistical computation on a signal specified as the second signal.

8. The biological signal estimation device according to claim 1, wherein
the first and second measurement devices measure the first and second signals including a pulse signal and a motion artifact signal generated in the living body, and
the processor is configured to execute the computer program to estimate the pulse signal as the biological signal.

9. The biological signal estimation device according to claim 1, wherein the processor is configured to execute the computer program to analyze a state of the living body based on the biological signal estimated.

10. The biological signal estimation device according to claim 9, wherein the processor is configured to execute the computer program to analyze an emotion of the living body.

11. The biological signal estimation device according to claim 9, wherein the processor is configured to execute the computer program to analyze an abnormal point in the living body.

12. The biological signal estimation device according to claim 11, wherein the processor is configured to execute the computer program to specify, as the abnormal point, a point where the first measurement means related to the biological signal whose characteristic is changed among a plurality of the biological signals is arranged, the plurality of the biological signals being estimated based on measurement results of a plurality of the first measurement means arranged in a vicinity of a blood vessel of the living body.

13. A biological signal estimation method comprising:
measuring, by a first measurement device, a first signal generated in a living body;
measuring, by a second measurement device, a second signal generated in the living body;
comparing, by an information processing device, characteristics of the first and second signals; and
estimating, by the information processing device, a biological signal in the living body by performing signal processing on the first and second signals based on a result of the comparison of the characteristics.

14. A non-transitory computer-readable recording medium storing a biological signal estimation program for causing a computer to implement:
a comparison function of comparing characteristics of a first signal measured by first measurement means and a second signal measured by second measurement means, the first and second signals being generated in a living body; and
an estimation function of estimating a biological signal in the living body by performing signal processing on the first and second signals based on a result of the comparison of the characteristics obtained by the comparison function.
